SEQUENCE LISTING (9 sequences)

SEQ ID NO: 1 - protein, 405 aa, C. Difficile
MQDYKKNKRRMMNQPMSTMNEEEVYTDEINSEDMRGFKKSHHHNGCNTDNKCECHDDCNPCNPCNPCKPNPCNPCKPNPCDDNCGCHDNCKCDCEPCEMDSDECFENKCGPECCNPISPRNFSVSNAVPFAIEANRIFDTMQFQTFTDATGPNGEPLTFETEVVEVFGSVPSAGQASVTIEKICLSNDGIVIDTGMTTLEDFDLDPLGDIVGRNCETTFEFAVCGERNSECCRQGKGKSVAYKQRGLTVAVRNLVLELRGRCGCTEFVALAFPAVRAGGGCKRRVDYVEFTFNTLSAPICLPADGRAVTLRQEYQTNLTVDCIGKSILKLECNECCEPFYELIIPNDIDLVLCLQETVSTLISEQIVVLASPNPIQPRLVDTFSKVCDFSQCGPNHGSGKPSCHR

SEQ ID NO: 2 - protein, 693 aa, C. Difficile
MRNIILYLNDDTFISKKYPDKNFSNLDYCLIGSKCSNSFVKEKLITFFKVRIPDILKDKSILKAELFIHIDSNKNHIFKEKVDIEIKRISEYYNLRTITWNDRVSMENIRGYLPIGISDTSNYICLNITGTIKAWAMNKYPNYGLALSLNYPYQILEFTSSRGCNKPYILVTFEDRIIDNCYPKCECPPIRITGPMGPRGATGSTGPMGVTGPTGSTGATGSIGPTGPTGNTGATGSIGPTGVTGPTGSTGATGSIGPTGVTGPTGNTGVTGSIGPTGATGPTGNTGVTGSIGPTGVTGPTGNTGEIGPTGATGPTGVTGSIGPTGATGPTGEIGPTGATGATGSIGPTGATGPTGATGVTGEIGPTGEIGPTGATGPTGVTGSIGPTGATGPTGATGEIGPTGATGPTGVTGSIGPTGATGPTGATGEIGPTGATGPTGVTGEIGPTGATGPTGNTGVTGEIGPTGATGPTGNTGVTGEIGPTGATGPTGVTGEIGPTGNTGATGSIGPTGVTGPTGATGSIGPTGATGATGVTGPTGPTGATGNSSQPVANFLVNAPSPQTLNNGDAITGWQTIIGNSSSITVDTNGTFTVQENGVYYISVSVALQPGSSSINQYSFAILFPILGGKDLAGLTTEPGGGGVLSGYFAGFLFGGTTFTINNFSSTTVGIRNGQSAGTAATLTIFRIADTVMT

SEQ ID NO: 3 - protein, 423 aa, C. Difficile
MQDGFLTVSIIDATNNRPIQNAVVNIYSMSNGSQSSSTLYQNLRSNESGQVTGLVLPAPDVDYSLQPSDVRPYSQYIVEAIADGYETVVIEGTQLLATIEARQGVPMSPRTRSKRSFSRQSELIFDIGEHTLYGTYPPKIPESNLKPLPPPTGFVVLDNPVVPEFIVVHDGLPEDSSAPNYWIPFKEYIKNIASSEIYSTWPEQTIYANVIAIISFTLNRVFTEWYRNKGYNFTITSTTAYDHKFINNRNLFEPINVVVDAIFNTFIKRPPTSRQPLLAQYCDGQKSQCPDQMTQWGSKDLGDQGYDYESILRYFYGDEIVFERAPIVSGVPVSFPGTTLQVGSSGQYVRTIQNQLNAISNSYPAVPKVIEDGIYGTDTENAVKIFQGIFGLPQSGVVDFKTWYEISRVYVATTRIASLNPLI

SEQ ID NO: 4 - protein, 305 aa, C. Difficile
MENNKCREDFRFTQEYEEDYPNTNERYYENYQVADRYYNYPNKYKEPKIKQCCCKKSMREALELLRYDALRPFVNFNQFAFISDFFIVGANLVGIDLSAPPKDNLSGLDGTFERFSACNCDLIDIAGRVSYPIPVPLTLEGLINTIGTIPGVAELIALIDAVIPPTIDLGAILDAILAAIIDFILAASTPLANVDLASLCNLKAVAFDITPADYEDFIASLGYYLDKKHYKECNCNCDCDDCCCNKGILDNLYMSNINNQVTVVAGSLVLTGVEVLGKKNDVIVLGNSNDSRIYFVCVDSIDYIA

SEQ ID NO: 5 - protein, 160 aa, C. Difficile
MENKKCYSEDWYERGESTAKWFQNDREEYEREAYDEDRERRGSNCGCSDSGENRPRNCERFRREAEIREREAREAFCESSEKKKEALAYECEARKLWEEAEKYWDEYSKYNYKGIEYLAEAARLFDEGMECEARRNGNNGGNNNNCCHKCHKCNCNCCRK

SEQ ID NO: 6 - protein, 291 aa, C. Difficile
MRVNTNVSALIANNQMGRNVNAQSKSMEKLSSGVRIKRAADDAAGLAISEKMRAQIKGLDQAGRNVQDGISVVQTAEGALEETGNILQRMRTLSVQSSNETNTAEERQKIADELLQLKDEVERISSSIEFNGKKLLDGSSTEIRLQVGANFGTNVAGTSNNNNEIKVALVNTSSIMSKAGITSSTIASLNADGTSGTNAAKQMVSSLDVALKELNTSRAKLGAQQNRLESTQNNLNNTIENVTAAESRIRDTDVASEMVNLSKMNILVQASQSMLAQANQQPQGVLQLLGS

SEQ ID NO: 7 - protein, 507 aa, C. Difficile
MSSISPIRVTGLSGNFDMEGIIEASMIRDKEKVDKAKQEQQIVKWKQEIYRNVIQESKDLYDKYLSVNSPNSIVSEKAYSSTRITSSDESIIVAKGSAGAEKINYQFAVSQMAEPAKFTIKLNSSEPIVQQFPPNASGASSLTIGDVNIPISEQDTTSTIVSKINSLCADNDIKASYSEMTGELIISRKQTGSSSDINLRVIGNDNLAQQIANDNGITFINDAGGNKVANVYGKNLEADVTDEHGRVTHISKEQNSFNIDNIDYNVNSKGTAKLTSVTDTEEAVKNMQAFVDDYNKLMDKVYGLVTTKKPKDYPPLTDAQKEDMTTEEIEKWEKKAKEGILRNDDELRGFVEDIQSAFFGDGKNIIALRKLGINESENYNKKGQISFNADTFSKALIDDSDKVYKTLAGYSSNYDDKGMFEKLKDIVYEYSGSSTSKLPKKAGIEKTASASENVYSKQIAEQERNISRLVEKMNDKEKRLYAKYSALESLLNQYSSQMNYFSQAQGN

SEQ ID NO: 8 - protein, 2710 aa, C. difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 9 - protein, 2366 aa, C. Difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

(19) United States
(12) Patent Application Publication
(10) Pub. No.: US 2016/0250283 A1
(43) Pub. Date: Sep. 1, 2016

(54) COMPOSITIONS AND METHODS FOR PROPHYLAXIS AND THERAPY OF CLOSTRIDIUM DIFFICILE INFECTION

(71) Applicant: THE ROCKEFELLER UNIVERSITY, New York, NY (US)

(72) Inventors: Chandrabali GHOSE-PAUL, New York, NY (US); David H. HO, Chappaqua, NY (US)

(21) Appl. No.: 15/030,898

(22) PCT Filed: Oct. 23, 2014
(86) PCT No.: PCT/US2014/061896

§ 371 Date: Apr. 21, 2016

Related U.S. Application Data
(60) Provisional application No. 61/894,605, filed on Oct. 23, 2013.

(51) Int. Cl. A61K 38/16

(52) U.S. Cl. CPC A61K 38/164

(57) ABSTRACT

Provided are compositions and methods for prophylaxis and/or therapy of C. difficile infection, and for inhibiting dissemination of C. difficile spores. The compositions contain C. difficile proteins, including distinct proteins and fusions of C. difficile CD 1067, BclA1, SleC, CotA, Spl7, FliC, FliD, CD toxin A, CD toxin B, and combinations thereof. The methods include prophylaxis and/or therapy of C. difficile infection by administering to a subject in need a composition that includes the C. difficile protein(s).

### CROSS-REFERENCE TO RELATED APPLICATIONS

This application claims priority to U.S. Provisional Application No. 61/894,605, filed on Oct. 23, 2013, the disclosure of which is incorporated herein by reference.

### FIELD OF THE DISCLOSURE

This disclosure relates generally to infectious bacterial disease and more particularly to prophylaxis and/or therapy of Clostridium difficile infections.

### BACKGROUND

Clostridium difficile (C. difficile or “CD”) is a spore-forming Gram-positive anaerobic bacillus, and the leading cause of nosocomial infectious diarrhea and colitis in the industrialized world with more than 300,000 cases per annum in the U.S. Complications of C. difficile infection (CDI) include pseudomembranous colitis, toxic megacolon, systemic inflammatory response syndrome, and death. Broad-spectrum antibiotic usage, hospitalization, advanced age, and co-morbidities increase the risk of acquiring CDI. Although the spread of C. difficile spores may be reduced by strict adherence to hand hygiene and other contact precautions, such control practices are costly and have not yet yielded the desired results. Thus there is an ongoing and unmet need for new approaches for prophylaxis and/or therapy of CDI, as well as methods for inhibiting its persistence, particularly in patient-care settings, and its spread between individuals. The present disclosure addresses these needs.

### SUMMARY OF THE DISCLOSURE

The present disclosure provides compositions and methods for prophylaxis and/or therapy of Clostridium difficile (C. difficile) infection. In one aspect, the disclosure includes a pharmaceutical composition comprising at least one C. difficile polypeptide for use in vaccinating against and/or for therapy of a C. difficile infection. The polypeptide can be selected from the group consisting of C. difficile CD1067, BclA1, SleC, CotA, Spl7, and combinations thereof. In one embodiment, the composition comprises CD1067.

In another aspect fusion proteins are provided. The fusion proteins can comprise any combination of at least two C. difficile proteins selected from the group consisting of CD1067, BclA1, SleC, CotA, Spl7, FliC, FliD, CD toxin A (CDA), CD toxin B (CDB), and combinations thereof. In embodiments, the fusion proteins comprise a combination of at least two of CD1067, BclA1, SleC, CotA, and Spl7.

In embodiments the disclosure includes a method for prophylaxis and/or therapy of C. difficile infection comprising administering to a subject in need thereof an effective amount of a pharmaceutical composition disclosed herein. The administration can be such that the C. difficile infection is prevented, or the amount of C. difficile in the subject is reduced, or one or more of the following is inhibited or prevented: diarrhea, including but not necessarily limited to antibiotic-associated diarrhea (AAD); pseudomembranous colitis; toxic megacolon; systemic inflammatory response syndrome, CD infection relapse, and CD colonization of a subject. The subject can be any mammal. In embodiments, the subject is a human. In other embodiments, the subject is a non-human mammal and as such the compositions and methods are suitable for veterinary approaches to combatting C. difficile infection.

In embodiments, articles of manufacture comprising at least one sealed container comprising at least one of the C. difficile polypeptides described herein and packaging, wherein the packaging comprises printed material providing an indication of the contents of the container and an indication that the product is for use in prophylaxis and/or therapy of C. difficile infection.

Expression vectors encoding polypeptides, and particularly fusion proteins described herein are provided, as are methods of using the expression vectors for producing the polypeptides.

### BRIEF DESCRIPTION OF THE DRAWINGS

FIG. 1 provides a graphical representation of data showing serum anti-Cd1067 IgG responses in mice immunized with CD1067 adjuvanted with alum.

FIG. 2 provides a graphical representation of data showing survival in CD1067 vaccinated C57BL/6 mice following challenge with strain UK1 C. difficile spores.

FIG. 3 provides a graphical representation of data showing survival in CD1067 vaccinated C57BL/6 mice following challenge with UK1 C. difficile spores.

FIG. 4 provides a graphical representation of data summarizing anti-CD1067 IgG responses in serum of mice 1 day before challenge.

FIG. 5 provides a graphical summary of data showing weights of mice following orogastric challenge with 10⁶ cfu of strain UK1 C. difficile spores.

FIG. 6 provides a graphical summary of data showing spore protein immunogenicity in mice using three distinct CD proteins. The panels shown serum anti-Cd1067 IgG (panel a), anti-SleC IgG (panel b) and anti-BclA1 IgG (panel c) responses in mice.

FIG. 7 provides a schematic diagram of representative CD fusion protein constructs using CD FliC, FliD, CDA and CDB toxoid fragments as examples. Those skilled in the art will recognize based on the present disclosure that any of the proteins described herein can be substituted for and/or combined with the FliC and FliD polypeptides and CDA and/or CDB domains.

FIG. 8A provides a graphical summary of data showing analysis of immunogenicity of CD FliC and FliD in mice. FIG. 8B provides a graphical summary of data showing analysis of serum anti-CDA IgG (a), anti-CDB IgG (b) and anti-FliC IgG (c) responses in mice.

FIG. 9 provides a graphical summary of data showing that CD fusion protein constructs are immunogenic.

FIG. 10 also provides a graphical summary of data showing that CD fusion protein constructs, as well as individual CD proteins, are immunogenic.

FIG. 11 provides a graphical depiction of results demonstrating survival in vaccinated C57BL/6 mice following orogastric challenge with 10⁶ cfu of strain UK1 C. difficile spores.

FIG. 12 provides a graphical summary of data demonstrating survival in vaccinated C57BL/6 mice following orogastric challenge with 10⁶ cfu of strain UK1 C. difficile spores.

FIG. 13 provides a graphical summary of data showing anti-FliC IgG responses in serum of mice 1 day before challenge.

FIG. 14 provides a graphical summary of data summarizing weights of mice following orogastric challenge with 10⁶ cfu of strain UK1 C. difficile spores.

FIG. 15 provides a graphical summary of data summarizing Immunogenicity and protective efficacy of C. difficile spore coat proteins. (a), Serum anti-CotA IgG and anti-Spl7 IgG responses in mice immunized on days 0, 14 and 28. Cohorts of mice received 25 ug of each antigen adjuvanted with alum. Results were determined by kinetic ELISA and are reported as optical density (OD) units; the geometric mean plus standard error of the mean for each cohort is shown. (b) Survival in vaccinated C57BL/6 mice following orogastric challenge with 10⁶ cfu of strain UK1 C. difficile spores.

### DETAILED DESCRIPTION

The present disclosure relates to compositions and methods for prophylaxis and/or or therapy of C. difficile (CD) infection and is expected to provide the additional benefit of reducing dissemination and/or carriage of CD spores.

In general the compositions and methods relate to CD polypeptides and/or immunogenic fragments and/or derivatives of the polypeptides, and include CD fusion proteins and combinations of distinct CD proteins. The compositions generally comprise pharmaceutical preparations comprising one or more CD proteins and the methods generally comprise administering to a subject an effective amount of such a pharmaceutical preparation so that CD infection of the subject is inhibited, reduced or prevented. In embodiments, carriage and/or dissemination of CD spores is inhibited, reduced or prevented. In particular embodiments the disclosure provides fusion proteins comprising the C. difficile CD1067 protein (also referred to in the art as “CdeC”) or immunogenic fragments or derivatives of it, and combinations of such CD1067 proteins and other CD proteins.

CD1067 has been recently described as having poor antigenic properties (see, Barra-Carrasco et al., J. of Bacteriology, (2013) Vol. 105, pp 3863-3875). Further, while CD1067 has been suggested as a marker for detecting the presence of C. difficile spores using an antibody preparation directed to spore surface proteins (U.S. patent publication no. 20100291100, published Nov. 18, 2010), it was not used in a vaccine context, and was not shown to generate neutralizing antibodies or to have any protective effect against C. difficile infection.

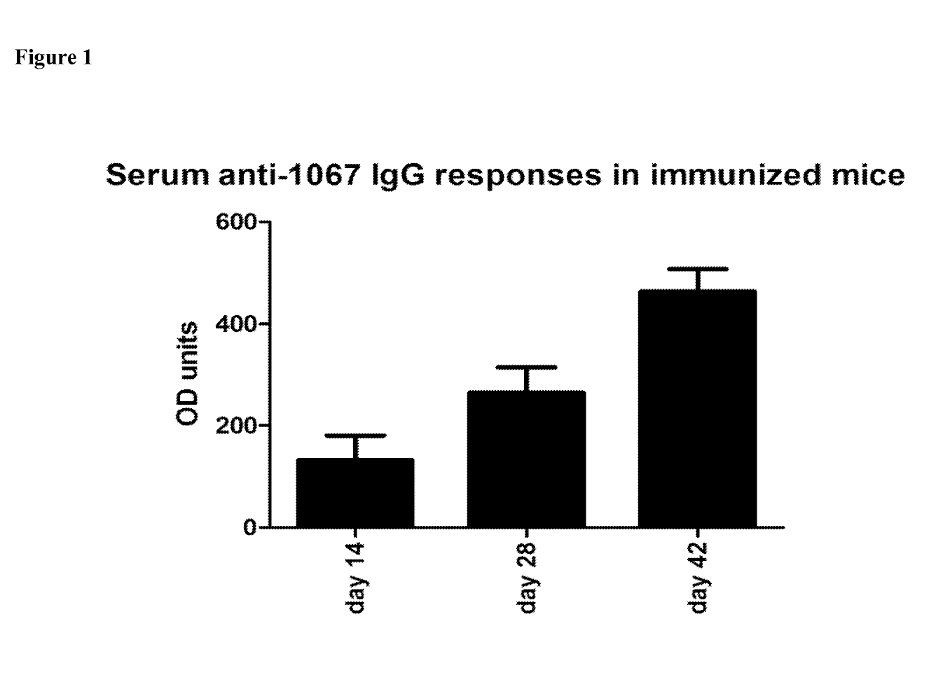

In contrast, the present disclosure includes the demonstrations that the CD1067 protein is not only an effective immunogen which can stimulate a robust anti-CD1067 IgG responses in mice, but can also inhibit CD-infection induced weight loss, CD-induced diarrhea and can moreover extend survival of mice after challenge with CD spores (see FIGS. 1-6), which were unexpected results. Thus, the present disclosure provides a method that is surprisingly protective against CD infection, diarrhea and death.

It is expected that any CD protein or immunogenic fragment of it obtained and/or derived from any C. difficile isolate or culture as further described herein will be suitable for use with embodiments of the present disclosure. In one non-limiting example, a CD1067 protein having the amino acid sequence disclosed under National Center for Biotechnology Information (NCBI) GeneID YP_001087551 (SEQ ID NO:1) is used.

For each amino acid sequence described herein the disclosure includes all amino acid sequences having between 90% and 99% similarity, inclusive and including each digit there between, with the amino acid sequence provided for the protein in its sequence identifier. Variations in sequence can comprise conservative or non-conservative amino acid substitutions, insertions, and deletions and protein fragments, provided the variant protein retains or improves an immunostimulatory function of the non-variant protein. In embodiments, a variant CD polypeptide used in a composition or method of the disclosure has sufficient identity with a wild type sequence such that the variant is specifically recognized by an antibody that specifically recognizes the wild type protein.

The disclosure includes all polynucleotide sequences encoding each amino acid sequence and variant described herein, including mRNA, DNA and cDNA sequences. The polynucleotides can be provided in any of a variety of recombinant molecules, such as plasmids, shuttle vectors, and expression vectors.

The amino acid sequences of the proteins referenced in this disclosure are known in the art and are provided in the sequence listing that follows this description and examples. In addition to CD1067, other CD proteins that can be used include but are not necessarily limited to GenBank No. YP_001087551 (SEQ ID NO:1), BclA1 (SEQ ID NO:2; GenBank No. YP_001086801.1), SleC (SEQ ID NO:3, GenBank No. YP_001087027.1), CotA (SEQ ID NO:4, GenBank No. YP_001088114.1, Spl7 (SEQ ID NO:5, GenBank No. YP_001088081.1) FliC SEQ ID NO: 6, GenBank No. AAD46086.1, FliD (SEQ ID NO:7, GenBank No. ZP_05349430, Toxin A (GenBank No. AAA23283.1) and Toxin B (GenBank No. P18177.3).

The polypeptides used in the present disclosure can be encoded by any strain of C. difficile, including but not necessarily limited to CD strain 630, strain UK1, or strain VPI 10463, or any other strain. In embodiments, a sample of CD can be obtained from a subject patient or location and cultured, and any CD protein encoded by the CD culture can be used to construct a CD composition according to the present disclosure. Such constructs could be used for instance, for patient and/or location specific approaches for combating CD infection and its dissemination within a distinct patient population.

In embodiments the present disclosure includes compositions and/or fusion proteins which contain any combination of polypeptide sequences obtained or derived from any of the following CD proteins: CD1067, BclA1, SleC, CotA, Spl7, FliC, FliD, CDA, and CDB. In embodiments, the disclosure includes a pharmaceutical composition comprising any combination of CD1067, BclA1, SleC, CotA, Spl7 polypeptides.

The compositions can comprise the CD1067, BclA1, SleC, CotA, Spl7, and combinations thereof as distinct proteins molecules, or they can be components of the same polypeptide in a fusion protein. In embodiments, at least two of the polypeptides are provided as components of a fusion protein.

In one embodiment, the CD1067 polypeptide is provided as a component of a fusion protein with at least one polypeptide sequence selected from the sequences of BclA1, SleC, CotA, Spl7, FliC, FliD, CDA, CDB, and combinations thereof. Thus, the disclosure includes a variety of multivalent vaccine formulations with respect to CD epitopes. In embodiments the disclosure includes a fusion protein comprising a combination of a CD1067 polypeptide and a BclA1 polypeptide, or a CD1067 polypeptide and a SleC polypeptide, or a polypeptide CD1067 and a CotA polypeptide, or a CD1067 polypeptide and a Spl7 polypeptide, or a CD1067 polypeptide and a FliC polypeptide, or a CD1067 polypeptide and a FliD polypeptide, or a CD1067 polypeptide and CDA polypeptide, or a CD1067 and a CDB polypeptide. In embodiments, a fusion protein comprising a CD1067 polypeptide includes more than one other CD polypeptide. In embodiments, a fusion protein comprises at least two of BclA1, SleC, CotA, and Spl7 polypeptides.

With respect to toxins, C. difficile expresses two major virulence factors, toxin A (CDA) and toxin B (CDB). These large exotoxins (CDA, 308 kDa; CDB, 270 kDa) have a tripartite structure with an enzymatically active N-terminal domain, a central translocation section and a C-terminal receptor-binding domain (RBD) consisting of repeating units of 21, 30 or 50 amino acid residues. In embodiments, only the RBD domains or immunogenic fragments thereof from the CD toxins are used in the compositions and methods of the present disclosure.

In general, proteins used in the compositions and methods of this disclosure will include full length or segments of CD proteins. In embodiments, a segment of CD1067 (or any other CD protein) will be of sufficient length to include at least one immunostimulatory epitope and in general will comprise a contiguous segment of at least 15 amino acids of the CD protein. Thus, the disclosure includes polypeptides which comprise at least 15 contiguous amino acids of CD1067, and also include fusion proteins that further comprise at least 15 contiguous amino acids from at least one of the other CD proteins described herein. There is no particular limitation as to how fusion proteins can be configured in that, in embodiments, CD1067 amino acids can be provided at the N-terminus, the C-terminus, or between the N- and C-termini of any particular polypeptide. The fusion protein can have another CD polypeptide N-terminal or C-terminal to a CD1067 sequence, or a CD1067 amino acid sequence can be flanked by other CD polypeptide sequences.

In embodiments, any polypeptide of the disclosure can have modifications which comprise amino acids designed to facilitate production of the proteins using recombinant expression systems. For example, the proteins can be provided with a leader or secretory sequence, or a sequence that aids in purification of the fused polypeptide, such as a hexa-histidine peptide.

In embodiments, the instant disclosure provides pharmaceutical preparations which comprise one or more CD polypeptides described herein. The pharmaceutical preparations comprise an additive, such as a diluent, adjuvant, excipient, or carrier. Such additives can be liquids, such as water and oils, saline, and auxiliary, stabilizing, thickening, or lubricating agents, wetting or emulsifying agents, or pH buffering agents. The pharmaceutical preparations can be used for prophylaxis and/or therapy, and in either case can be considered a vaccine formulation.

In an embodiment, the disclosure includes an article of manufacture which contains packaging and at least one of the CD1067, BclA1, SleC, CotA, or Spl7 polypeptides, or any combination thereof in a sealed container. The packaging contains printed material which provides an indication that the contents of the package is to be used for prophylaxis or treatment of CD infection. The container in embodiments comprises an isolated and/or recombinantly produced polypeptide, and thus does not contain CD whole cells. In embodiments, a CD1067 polypeptide is included. In embodiments, any one of the polypeptides disclosed herein may be the only CD polypeptide provided with the article of manufacture. In an embodiment, CD1067 is the only CD polypeptide provided. In embodiments the printed material is provided on the packaging material itself, or on a label, paper insert, etc. In embodiments the printed material identifies the CD polypeptide(s) that are included with the article, and provides an indication that the composition is to be used for reducing or preventing one or more symptoms of CD infection, including but not necessarily limited to CD-induced diarrhea.

The present compositions can be provided in solutions, suspensions, emulsions, tablets, pills, sustained-release formulations, suppositories, emulsions, aerosols, sprays, suspensions, or any other form suitable for introducing the compositions into an subject to stimulate an immune response against CD.

Methods of the disclosure include prophylactic and therapeutic approaches. Generally, prophylaxis includes introducing a composition of the invention into a subject before CD infection, or before symptoms or other indicia of CD infection are apparent. Therapeutic approaches includes introducing a composition of the invention into a subject after CD infection or symptoms or other indicia of CD infection are apparent.

The compositions can be introduced into a subject using any suitable administration route, including but not necessarily limited to parenteral, subcutaneous, intraperitoneal, intramuscular, intravenous, intraarterial, and oral administration.

Dosages, including the amount of CD protein and frequency of administration will be determined based on factors such as the age, health and size of the subject, as well as the subject's risk of contracting CD infection, or the stage of CD infection. In embodiments, a CD vaccine according to the present disclosure is administered in a single dose, or in sequential doses. The amount of CD protein can vary from, for example, 0.01-5,000 μg/ml, inclusive, and including all integers and ranges there between to the second decimal point, per dose. In embodiments, the dosage can range from 0.05 μg to 1,000 mg, inclusive, including all integers and ranges there between to the third decimal point.

In embodiments, the disclosure includes administering to an individual in need thereof an effective amount of a composition comprising a protein as described herein such that one or more of the following is inhibited or prevented: diarrhea, including but not necessarily limited to antibiotic-associated diarrhea (AAD); pseudomembranous colitis; toxic megacolon; systemic inflammatory response syndrome, CD infection relapse, and CD colonization of a subject. In embodiments, the subject is in need is a human. In embodiments, a subject to whom a composition of this disclosure is administered experiences less diarrhea relative to an individual who did not receive the pharmaceutical composition. In embodiments the subject may be at risk for C. difficile induced diarrhea. In embodiments, the subject may be infected with C. difficile.

In embodiments, the subject is a non-human animal, such as a non-human mammal. In embodiments, the non-human mammal is a porcine, canine, feline, equine, avian, or bovine mammal. In embodiments, the non-human mammal is a ruminant. Thus, the disclosure includes veterinary formulations and methods of administering the compositions to non-human animals. In embodiments, the individual to whom a composition is administered is in need thereof because the individual at risk for developing CD colonization and/CDI). In this regard, an individual who tests positive for CD or CD toxins in stool, but exhibits no clinical symptoms is considered to be only CD colonized. An individual who tests positive for CD and exhibits clinical symptoms is considered to have CDI. An individual who is at risk for developing CD colonization or CDI can be any individual who is a patient in a health-care providing facility, such as a hospital, or a nursing home, or any other environment wherein the individual is likely to come into contact with CD spores. In embodiments the disclosure includes administering vaccines of the present disclosure to a plurality of subjects such that the dissemination of CD spores and/or transmission of CD infection between the subjects is reduced.

The CD proteins used in the compositions and method of the present disclosure can be produced using any suitable methods. In one approach, the proteins are separated from a CD culture and purified to any desired degree of purity. In another approach, the CD proteins are produced recombinantly. Recombinant methods include providing a recombinant expression vector encoding a protein or fusion protein described herein, introducing the expression into a suitable host microorganism culture, allowing expression of the protein or the fusion protein, and separating the protein or the fusion protein from the host microorganism culture. The disclosure thus encompasses expression vectors encoding all of the polypeptides disclosed herein, as well as the non-CD microorganisms into which such expression vectors have been introduced. Suitable expression vectors and microorganism hosts are known in the art and many are commercially available. In an embodiment, the host is E. coli. In an embodiment, the expression vector is a commercially available pET vector into which CD polypeptide-encoding sequences are inserted. Methods for purifying proteins for use in pharmaceutical preparations, including vaccines, are well known in the art and can be used for making compositions of the present disclosure.

Administration of compositions of the invention can be accompanied by use of any other compositions and methods used for combating CD infections, including but not necessarily limited to antibiotic treatments, such as with metronidazole, fidaxomicin or vancomycin, or other agents, such as cholestyramine, probiotic therapy, or passive immunotherapy.

The following examples are for illustration of particular embodiments of the disclosure and are not intended to be limiting.

### EXAMPLE 1

This Example provides a description of materials and methods used for making and testing polypeptides comprising CD1067 polypeptides.

Bacterial strains and media. Escherichia coli DH5 alpha was used for all subcloning steps and E. coli BL21DE3* was used for protein expression (Invitrogen, Carlsbad, Calif.). All strains were maintained at −70° C. in Luria Bertani (LB) medium containing 15% glycerol. LB medium contained ampicillin (100 μg/ml), kanamycin (50 μg/ml), X-Gal (5-bromo-4-chloro-3-indolyl-β-D-galactopyranoside) (80 μg/ml) or IPTG (isopropyl-beta-D-thiogalactopyranoside) (0.1 mM) were used (Sigma Aldrich, St. Louis, Mo.).

Genetic methods. Isolation of plasmid and bacterial chromosomal DNA, restriction enzyme digestion, agarose gel electrophoresis were performed using standard biological techniques. DNA restriction endonucleases, T4 DNA ligase, and calf intestinal alkaline phosphatase were used according to manufacturer's specifications (New England Biolabs, Beverly, Mass.). Enzyme digested products were separated on 1% agarose gel and extracted using QIAEX II Gel Extraction Kit (Qiagen, Valencia, Calif.).

Full length protein sequences were obtained for CD1067 from C. difficile strain 630 (ATCC BAA-1382). The corresponding nucleotides were synthesized (Blue Heron Biotechnologies, Bothell, Wash.).

Protein expression and purification. Cd1067 was cloned into expression vector, pET19b (Novagen). A histidine (His) tag was incorporated to facilitate purification. The expressed proteins were purified using Talon His-tag purification resin according to manufacturer's specifications (Clontech Laboratories Inc., Mountain View, Calif.). Protein was detected by gel electrophoresis and with electronblotting with anti-His tag antibody (Invitrogen). Endotoxin was removed by using Endotrap Blue columns according to manufacturer's specifications (Hyglos GmbH, Bernried, Germany) Additional protein was commercially purified (Ascentgene, Rockville, Md.).

Immunization regimen. Female, 8- to 10-week-old, C57BL/6 mice were immunized on days 0, 14 and 28. Animal work was approved by the Institutional Animal Care and Use Committee at the Rockefeller University. In the first study, we immunized five cohorts of 5 mice each by intraperitoneal injection (I.P.) with 25 μg of CD1067 adjuvanted with or 1:1 by volume of Alum (Al(OH)3) (Sigma-Aldrich). We collected, processed, and stored blood samples from mice on days 0, 14, 28 and 42.

For the first challenge study, mice were immunized as described above. For the second challenge study, mice were either immunized with 5 ug or 25 ug of CD1067 and were either immunized on days 0 and 14 (2-shots), or were immunized on days 0, 14 and 28 (3 shots).

Measurement of immune responses. To detect antibody responses to CD1067, we coated plates with 100 ng/well of purified C. difficile CD1067 in 50 mM carbonate buffer, pH 9.6). We blocked plates with PBS-1% bovine serum albumin (BSA) (Sigma Aldrich). To detect anti-CD1067 IgG in serum, we diluted sera 1:1,000 in PBS containing 0.05% Tween 20 (PBS-T) (Sigma Aldrich), respectively, and incubated the plates at 37° C. for 1 h. We detected bound antibodies using a 1:1,000 dilution in PBS-T of goat anti-mouse IgG conjugated with horseradish peroxidase (HRP) (Southern Biotech, Birmingham, Ala.) incubating plates for 1 h at 37° C. We developed the plates with 2, 2′-azino-bis(3-ethylbenzthiazoline-6-sulfonic acid) (ABTS) (Sigma Aldrich) and 0.03% H₂O₂ (Sigma Aldrich) and determined optical density using a Vmax microplate reader (Molecular Devices Corp, Sunnyvale, Calif.) at 405 nm kinetically for 5 min at 14-second intervals. To equilibrate, we divided readings of milliunits of optical density per minute for samples by those for plate controls comprised of pooled blood or stool standards from unrelated experimental cohorts and reported the results as enzyme-linked immunosorbent assay (ELISA) units.

Mice challenge model. A murine model of antibiotic-associated CDI established recently in the Kelly laboratory and already used widely was used to evaluate the protective efficacy of our recombinant vaccine constructs. Female, 8- to 10-week-old, C57BL/6 mice were immunized on days 0, 14 and 28. Mice were administered a cocktail of antibiotics (kanamycin 40 mg/kg, gentamicin 3.5 mg/kg, colistin 4.2 mg/kg, metranidazole 21.5 mg/kg and vancomycin 4.5 mg/kg) in their water daily for 5 days starting on day 35. Two days later, mice received one dose of clindamycin (10 mg/kg) given intraperitoneally. 24 hours later, on day 42, mice were challenged with a dose of 10⁶ cfu of strain UK1 and monitored daily for 14 days for mortality, morbidity, weight and diarrhea. Animals that are moribund were euthanized.

### EXAMPLE 2

This Example provides a description of materials and methods used for making and testing polypeptides comprising individual and fusion CD proteins.

Bacterial strains and media. Bacteria strains and media are as described in Example 1.

Genetic methods. Isolation of plasmid and bacterial chromosomal DNA, restriction enzyme digestion, agarose gel electrophoresis were performed using standard biological techniques and as described in Example 1.

Full length protein sequences were obtained for CD1067 (SEQ ID NO:1, GenBank no. YP_001087551), BclA1 (SEQ ID NO:2; GenBank No. YP_001086801.1), SleC (SEQ ID NO:3, GenBank No. YP_001087027.1), CotA (SEQ ID NO:4, GenBank No. YP_001088114.1, Spl7 (SEQ ID NO:5, GenBank No. YP_001088081.1) FliC SEQ ID NO: 6, GenBank No. AAD46086.1, FliD (SEQ ID NO:7, GenBank No. ZP_05349430, Toxin A (GenBank No. AAA23283.1) and Toxin B (GenBank No. P18177.3)

Fusion constructs can be generated with FliC and FliD fused to CD1067, BclA1, SleC, CotA and Spl7 with FliC and FliD present at either terminus, or positioned elsewhere within the fusion peptide. Likewise, any of the fusion proteins described herein can have any of the polypeptides which are comprised by the fusion proteins positioned anywhere in the fusion protein, including but not limited to the N- or C-terminus.

Protein expression and purification. The orfs were cloned into expression vector, pET19b (Novagen). A histidine (His) tag was incorporated to facilitate purification. The expressed proteins were purified using Talon His-tag purification resin according to manufacturer's specifications (Clontech Laboratories Inc., Mountain View, Calif.). Protein was detected by gel electrophoresis and with electronblotting with anti-His tag antibody (Invitrogen). Endotoxin was removed by using Endotrap Blue columns according to manufacturer's specifications (Hyglos GmbH, Bernried, Germany) Additional protein was commercially purified (Ascentgene, Rockville, Md.).

Immunization regimen. Female, 8- to 10-week-old, C57BL/6 mice were immunized on days 0, 14 and 28. Animal work was approved by the Institutional Animal Care and Use Committee at the Rockefeller University. In the first study, we immunized five cohorts of 5 mice each by intraperitoneal injection (I.P.) with 25 μg of each protein adjuvanted with or 1:1 by volume of Alum (Al(OH)3) (Sigma-Aldrich). We collected, processed, and stored blood samples from mice on days 0, 14, 28 and 42.

For the first challenge study, mice were immunized as described above. For the second challenge study, mice were either immunized with 5 ug or 25 ug of each recombinant protein and were either immunized on days 0 and 14 (2-shots), or were immunized on days 0, 14 and 28 (3 shots).

Measurement of immune responses. To detect antibody responses to each protein, we coated plates with 100 ng/well of each purified C. difficile protein in 50 mM carbonate buffer, pH 9.6 (We blocked plates with PBS-1% bovine serum albumin (BSA) (Sigma Aldrich). To detect anti-C. difficile protein-IgG in serum, we diluted sera 1:1,000 in PBS containing 0.05% Tween 20 (PBS-T) (Sigma Aldrich), respectively, and incubated the plates at 37° C. for 1 h. We detected bound antibodies using a 1:1,000 dilution in PBS-T of goat anti-mouse IgG conjugated with horseradish peroxidase (HRP) (Southern Biotech, Birmingham, Ala.) incubating plates for 1 h at 37° C. We developed the plates with 2,2′-azino-bis(3-ethylbenzthiazoline-6-sulfonic acid) (ABTS) (Sigma Aldrich) and 0.03% H₂O₂ (Sigma Aldrich) and determined optical density using a Vmax microplate reader (Molecular Devices Corp, Sunnyvale, Calif.) at 405 nm kinetically for 5 min at 14-second intervals. To equilibrate, we divided readings of milliunits of optical density per minute for samples by those for plate controls comprised of pooled blood or stool standards from unrelated experimental cohorts and reported the results as enzyme-linked immunosorbent assay (ELISA) units.

Mice challenge model. A murine model of antibiotic-associated CDI established recently in the Kelly laboratory and already used widely was used to evaluate the protective efficacy of our recombinant vaccine constructs. Female, 8- to 10-week-old, C57BL/6 mice were immunized on days 0, 14 and 28. Mice were administered a cocktail of antibiotics (kanamycin 40 mg/kg, gentamicin 3.5 mg/kg, colistin 4.2 mg/kg, metranidazole 21.5 mg/kg and vancomycin 4.5 mg/kg) in their water daily for 5 days starting on day 35. Two days later, mice received one dose of clindamycin (10 mg/kg) given intraperitoneally. 24 hours later, on day 42, mice were challenged with a dose of 10⁶ cfu of strain UK1 and monitored daily for 14 days for mortality, morbidity, weight and diarrhea. Animals that are moribund were euthanized.

### EXAMPLE 3

This Example demonstrates production of serum anti-CD1067 IgG responses in mice using a pharmaceutical composition comprising CD1067 and alum. Results shown in FIG. 1 were determined by kinetic ELISA and are reported as ELISA units; the geometric mean plus standard error of the mean for each cohort is shown. It is apparent that, in contrast to recently published literature, CD1067 can be used to stimulate an antibody response.

### EXAMPLE 4

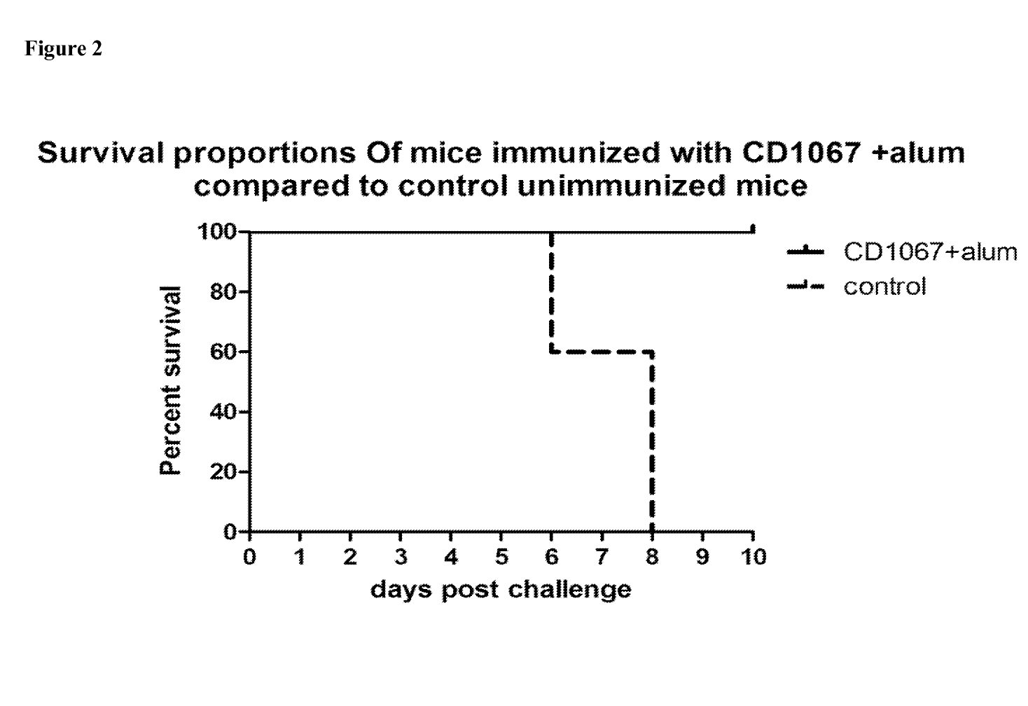

This Example demonstrates enhancement of survival of mice which received a CD1067 vaccine and were subsequently challenged with CD spores. In particular, the data in FIG. 2 show survival in vaccinated C57BL/6 mice following orogastric challenge with 10⁶ cfu of strain UK1 C. difficile spores. Mice were immunized with 25 ug of CD1067 adjuvanted with alum. Control mice were immunized with saline. Mice were immunized on day 0, 14 and 28 and challenged on day 42 following antibiotic treatment (orally administered antibiotic cocktails on day 35 for 5 days, intraperitoneally administered clindamycin on day 41.)

### EXAMPLE 5

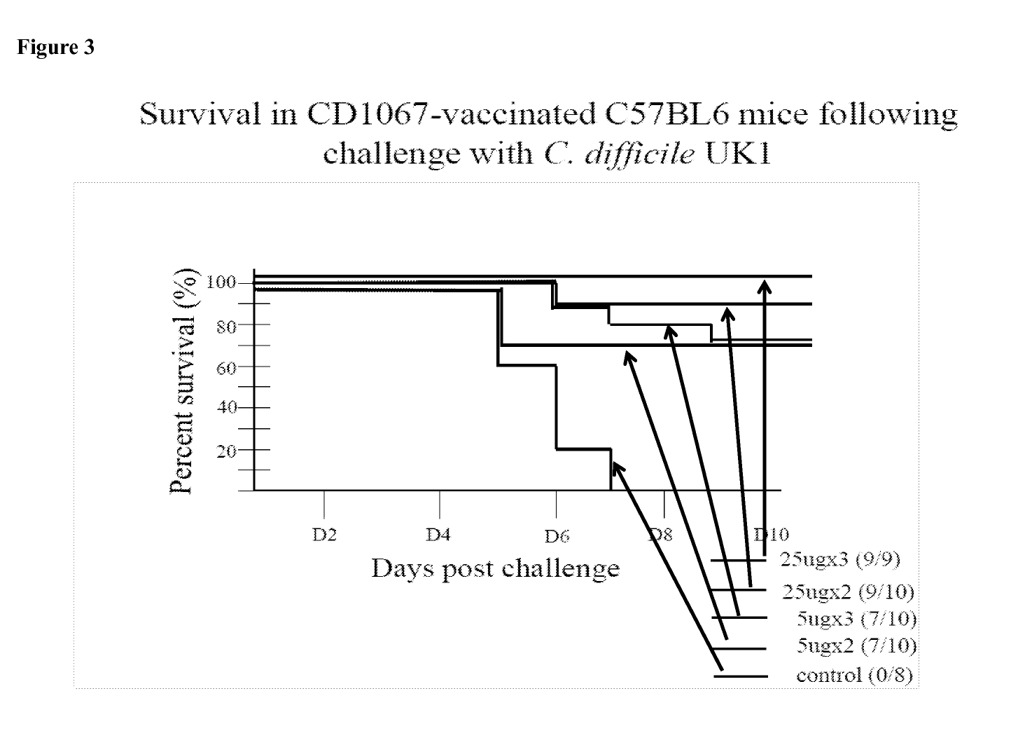

This Example expands on Example 4 and further demonstrates that vaccination with a composition comprising CD1067 results in survival after challenge with CD spores. In particular, that data presented in FIG. 3 are from C57BL/6 mice which received orogastric challenge with 10⁶ cfu of strain UK1 C. difficile spores. Cohorts of mice received either 5 ug or 25 ug total of CD1067 adjuvanted with alum. Control mice were immunized with saline. Mice were immunized on day 0, 14 and 28 (3 immunizations) or on days 0, and 14 (2 immunizations) and challenged two weeks post last immunization following antibiotic treatment (orally administered antibiotic cocktails for 5 days followed with intraperitoneally administered clindamycin 1 day before challenge.)

### EXAMPLE 6

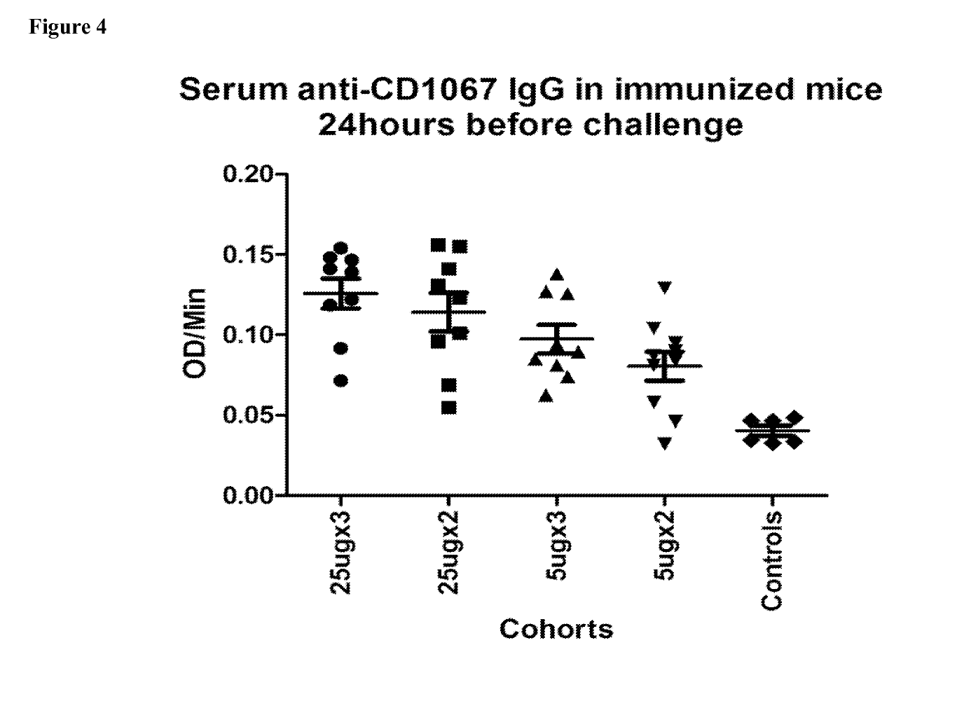

This Example demonstrates anti-CD1067 IgG responses in serum of mice 1 day before challenge. Cohorts of mice received either 5 ug or 25 ug total of CD1067 adjuvanted with alum. Control mice were immunized with saline. Mice were immunized on day 0, 14 and 28 (3 immunizations) or on days 0, and 14 (2 immunizations) and challenged two weeks post last immunization following antibiotic treatment (orally administered antibiotic cocktails for 5 days followed with intraperitoneally administered clindamycin 1 day before challenge.) Results presented in FIG. 4 were determined by kinetic ELISA and are reported as ELISA units; the geometric mean plus standard error of the mean for each cohort is shown.

### EXAMPLE 7

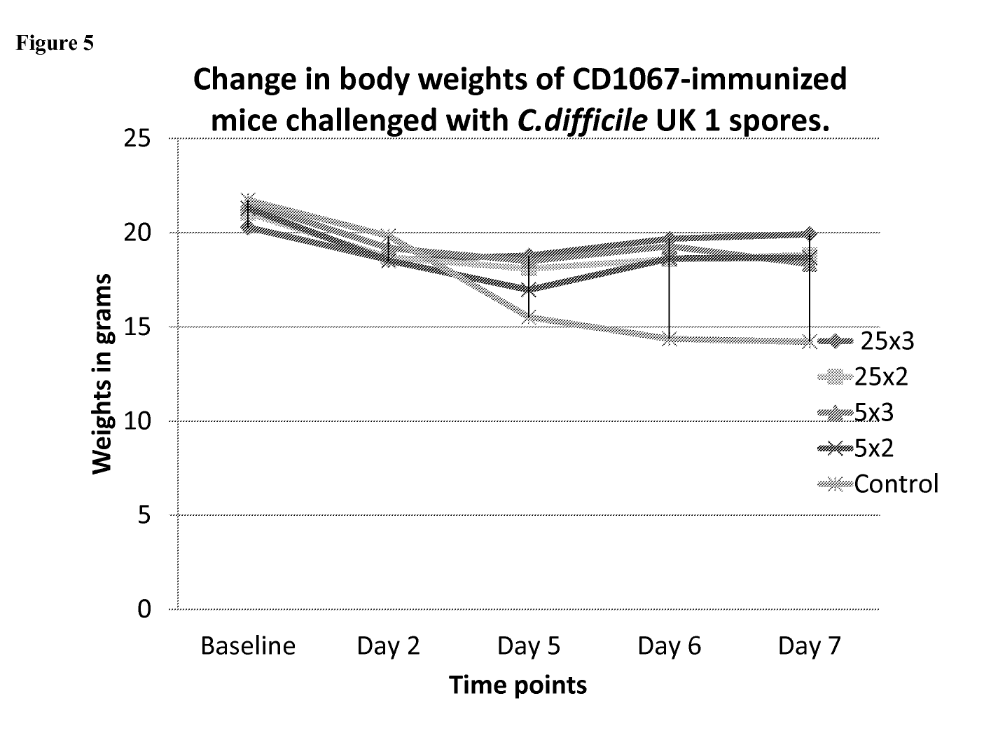

This Example demonstrates the effect of vaccination on weight of mice after challenge with CD spores. The data presented in FIG. 5 show weights of mice following orogastric challenge with 10⁶ cfu of strain UK1 C. difficile spores. Cohorts of mice received either 5 ug or 25 ug total of CD1067 adjuvanted with alum. Control mice were immunized with saline. Mice were immunized on day 0, 14 and 28 (×3 immunizations) or on days 0, and 14 (×2 immunizations) and challenged two weeks post last immunization following antibiotic treatment (orally administered antibiotic cocktails for 5 days followed with intraperitoneally administered clindamycin 1 day before challenge.) It is apparent from FIG. 5 that vaccination inhibits CDI-induced weight loss.

### EXAMPLE 8

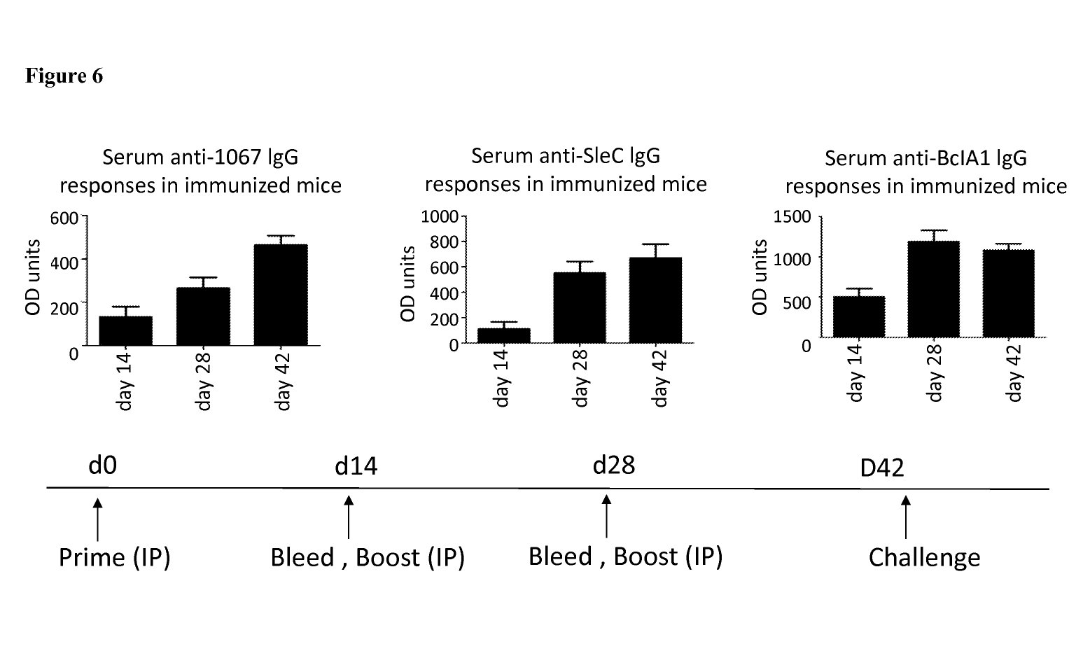

This Example demonstrates stimulation of an Ig response in mice using three distinct CD proteins. The panels in FIG. 6 show serum anti-CD1067 IgG (panel a), anti-Slec IgG (panel b) and anti-BclA1 IgG (panel c) responses in mice immunized on days 0, 14 and 28 Cohorts of mice received 25 ug of CD1067, BclA1 or SleC adjuvanted with alum. Results were determined by kinetic ELISA and are reported as ELISA units; the geometric mean plus standard error of the mean for each cohort is shown.

### EXAMPLE 9

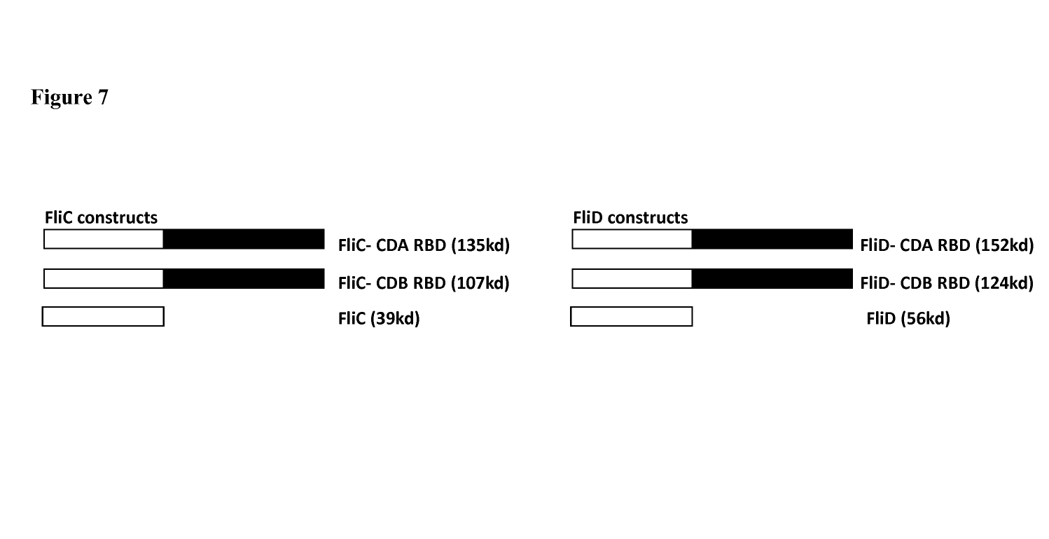

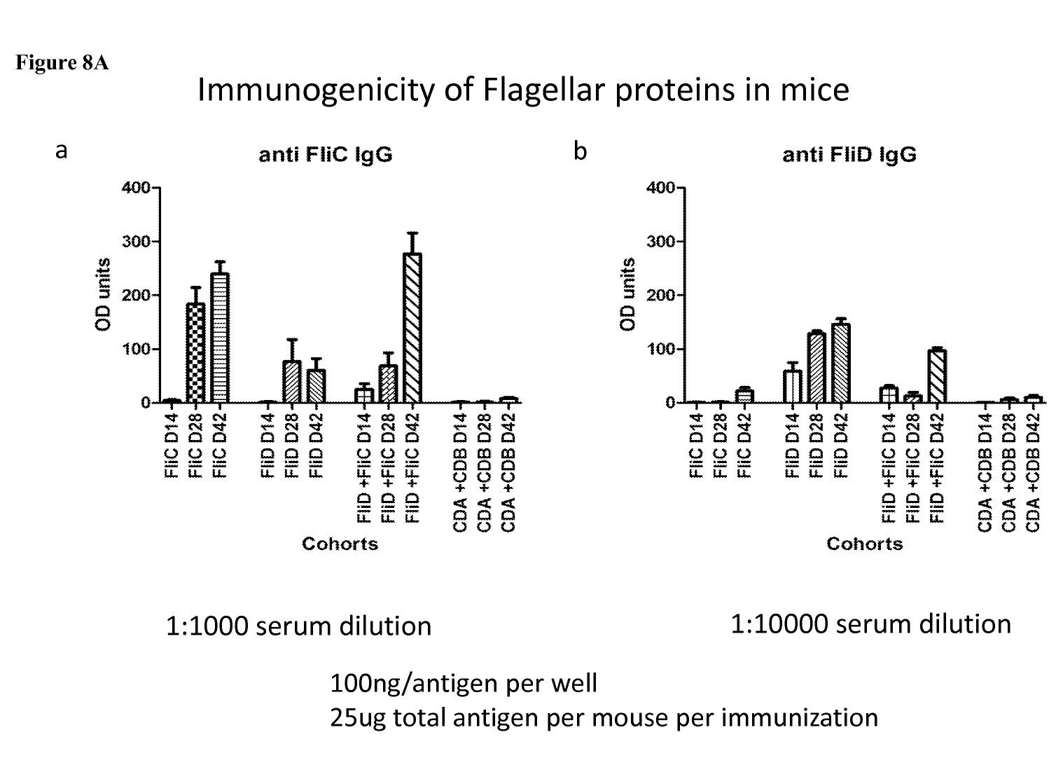

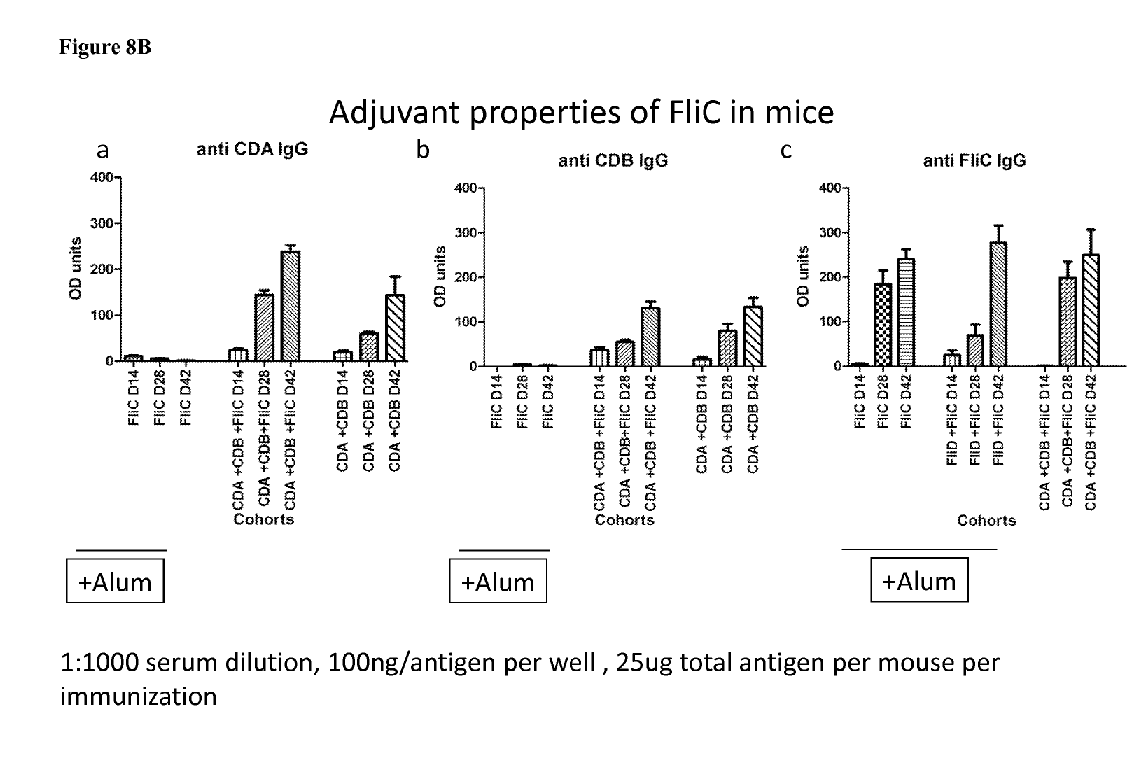

This Example demonstrates immunogenicity of various CD proteins. FIG. 7 provides a schematic diagram of representative CD fusion protein constructs using CD FliC, FliD, CDA and CDB toxoid fragments. The data in FIG. 8A show immunogenicity of CD FliC and FliD and in particular demonstrate production of serum anti-FliC IgG (a) and anti-FliD IgG (b) responses in mice immunized on days 0, 14 and 28 Cohorts of mice received 25 ug of FliC, FliD, or FliC and FliD together adjuvanted with alum. Cohorts also received 25 ug of CDA-RBD and CDB-RBD. Results were determined by kinetic ELISA and are reported as ELISA units; the geometric mean plus standard error of the mean for each cohort is shown.

The data in FIG. 8B show production of serum anti-CDA IgG (a), anti-CDB IgG (b) and anti-FliC IgG (c) responses in mice immunized on days 0, 14 and 28. Cohorts of mice received 25 ug of FliC, FliD, or FliC and FliD together adjuvanted with alum. Cohorts also received 25 ug of CDA-RBD and CDB-RBD, or 25 ug of CDA-RBD and CDB-RBD adjuvanted with FliC. Results were determined by kinetic ELISA and are reported as ELISA units; the geometric mean plus standard error of the mean for each cohort is shown.

### EXAMPLE 10

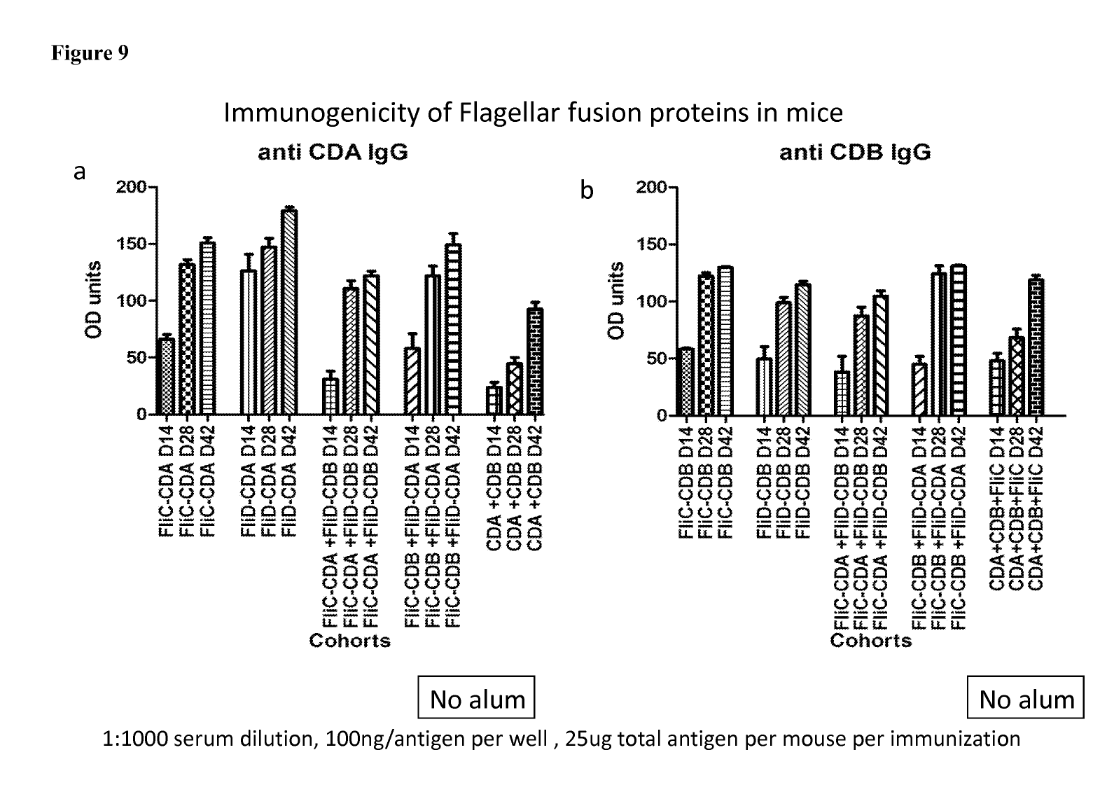

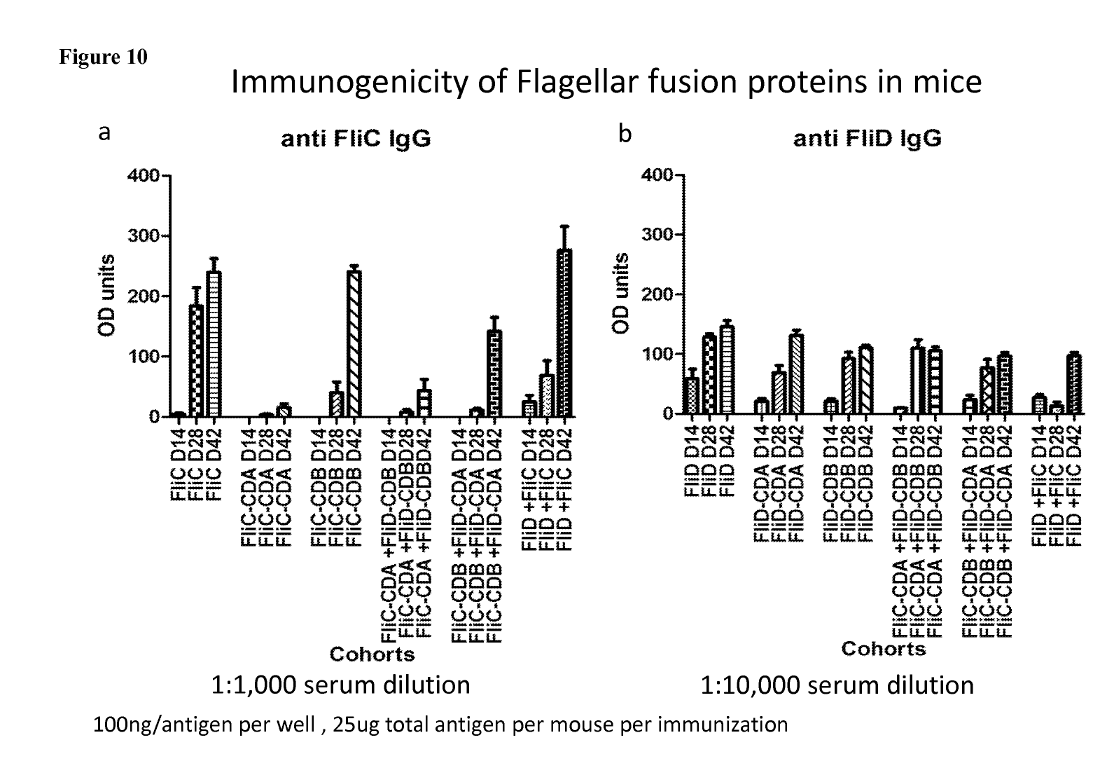

This Example demonstrates use of CD fusion proteins to stimulate anti-CD immune responses. In particular, the data in FIG. 9 show serum anti-CDA IgG (a) and anti-CDB IgG (b) responses in mice immunized on days 0, 14 and 28. Cohorts of mice received 25 ug total of fusion proteins FliC-CDA, FliC-CDB, FliD-CDA, FliD-CDB, FliC-CDA+ FliD-CDB, FliC-CDB+ FliD-CDA adjuvanted with alum. Cohorts also received 25 ug of CDA-RBD and CDB-RBD unadjuvanted. Results were determined by kinetic ELISA and are reported as ELISA units; the geometric mean plus standard error of the mean for each cohort is shown. The data presented in FIG. 10 also demonstrate generation of serum anti-FliC IgG (a) and anti-FliD IgG (b) responses in mice immunized on days 0, 14 and 28. Cohorts of mice received 25 ug total of fusion proteins FliC-CDA, FliC-CDB, FliD-CDA, FliD-CDB, FliC-CDA+ FliD-CDB, FliC-CDB+ FliD-CDA adjuvanted with alum. Results were determined by kinetic ELISA and are reported as ELISA units; the geometric mean plus standard error of the mean for each cohort is shown.

### EXAMPLE 11

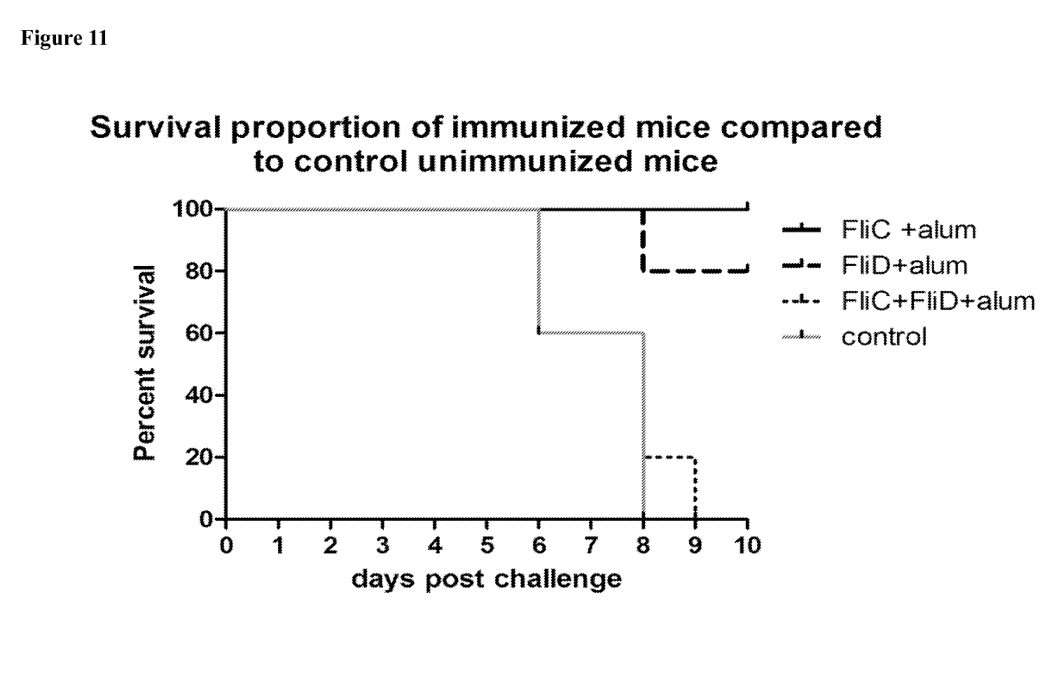

This Example demonstrates that vaccines comprising individual and CD fusion proteins can provide a prophylactic effect in mice challenged with CD spores. In particular, FIG. 11 provides a graphical depiction of results demonstrating survival in vaccinated C57BL/6 mice following orogastric challenge with 10⁶ cfu of strain UK1 C. difficile spores. Cohorts of mice received 25 ug total of FliC, FLiD adjuvanted with alum, FliC +FliD adjuvanted with alum, CDA+CDB+FliC, CDA+CDB+FliC+ FliD adjuvanted with alum, or with fusion proteins adjuvanted with alum, and CDA+ CDB unadjuvanted. Control mice were immunized with saline. Mice were immunized on day 0, 14 and 28 (3 immunizations) or on days 0, and 14 (2 immunizations) and challenged two weeks post last immunization following antibiotic treatment (orally administered antibiotic cocktails for 5 days followed with intraperitoneally administered clindamycin 1 day before challenge.)

### EXAMPLE 12

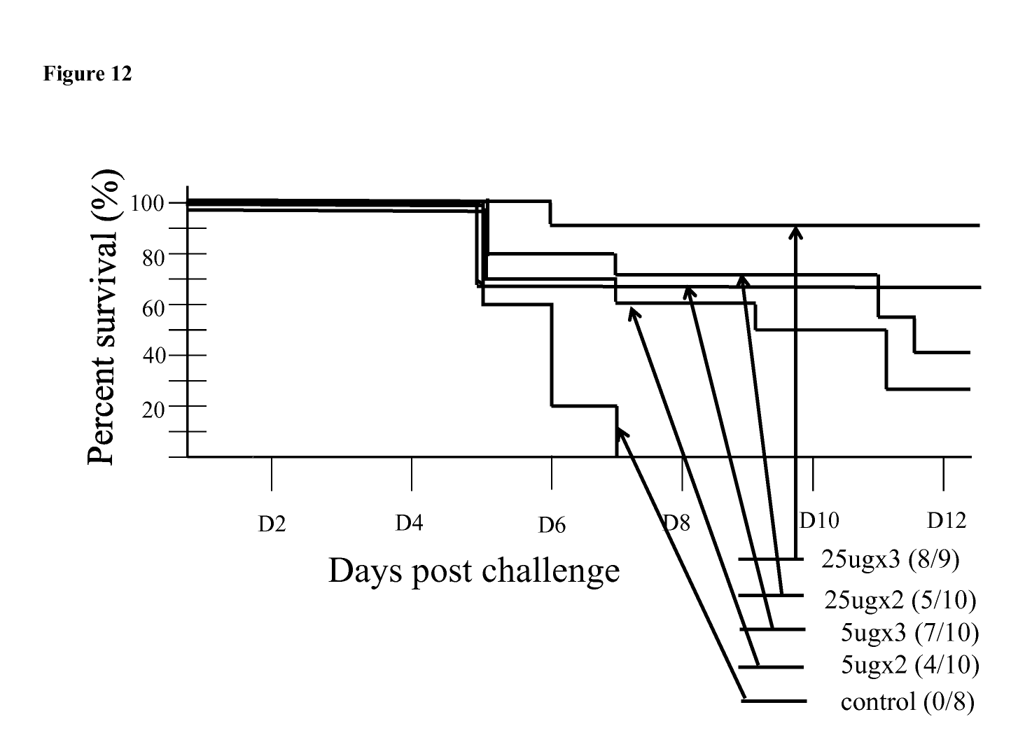

This Example demonstrates survival in FliC vaccinated C57BL/6 mice following orogastric challenge with 10⁶ cfu of strain UK1 C. difficile spores. FIG. 12 provides a summary of data from cohorts of mice which received either 5 ug or 25 ug total of FliC adjuvanted with alum. Control mice were immunized with saline. Mice were immunized on day 0, 14 and 28 (3 immunizations) or on days 0, and 14 (2 immunizations) and challenged two weeks post last immunization following antibiotic treatment (orally administered antibiotic cocktails for 5 days followed with intraperitoneally administered clindamycin 1 day before challenge.)

### EXAMPLE 13

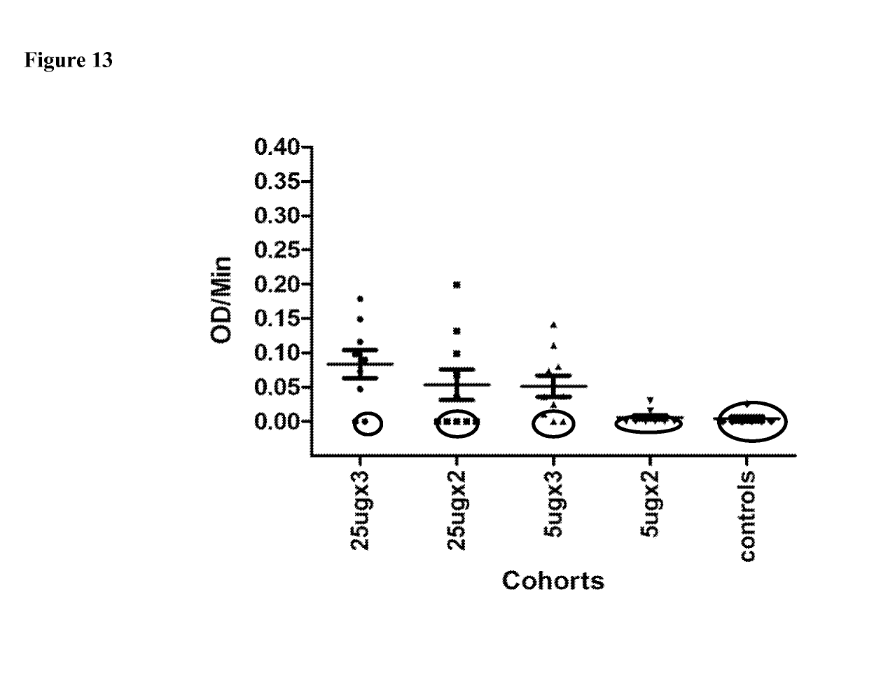

This Example demonstrates anti-FliC IgG responses in serum of mice 1 day before challenge. FIG. 13 provides a summary of data from cohorts of mice which received either 5 ug or 25 ug total of FliC adjuvanted with alum Mice were immunized on day 0, 14 and 28 (×3 immunizations) or on days 0, and 14 (×2 immunizations) and challenged two weeks post last immunization following antibiotic treatment (orally administered antibiotic cocktails for 5 days followed with intraperitoneally administered clindamycin 1 day before challenge.) Results were determined by kinetic ELISA and are reported as ELISA units; the geometric mean plus standard error of the mean for each cohort is shown. Data points circled denote mice that succumbed to challenge.

### EXAMPLE 14

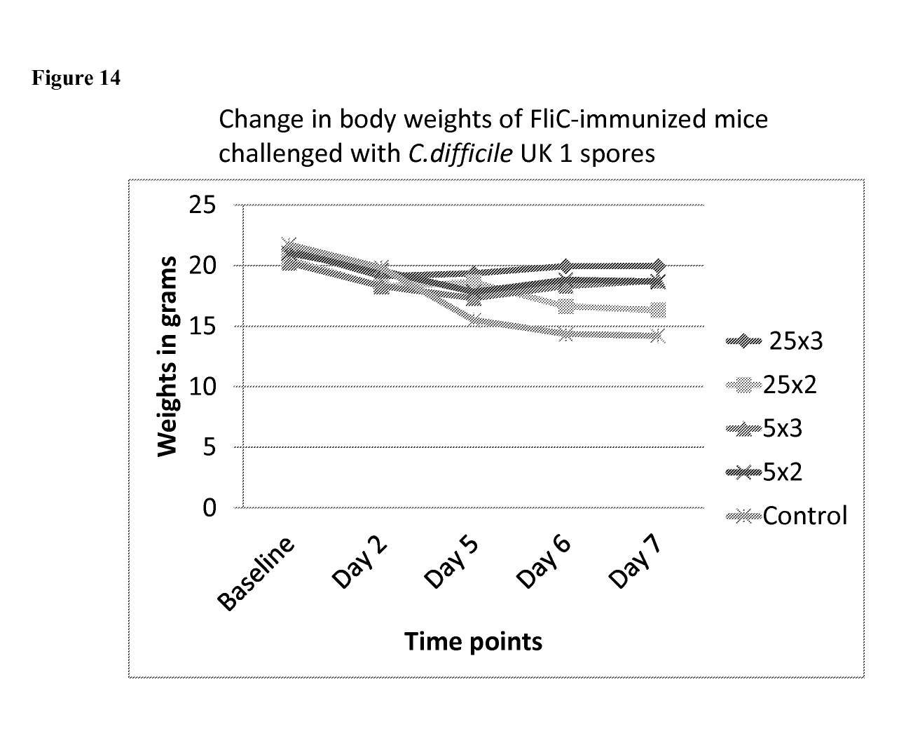

This Example shows a prophylactic effect against weight loss in mice following orogastric challenge with 10⁶ cfu of strain UK1 C. difficile spores. FIG. 14 provides a summary of data from cohorts of mice which received either 5 ug or 25 ug total of FliC adjuvanted with alum. Control mice were immunized with saline. Mice were immunized on day 0, 14 and 28 (×3 immunizations) or on days 0, and 14 (×2 immunizations) and challenged two weeks post last immunization following antibiotic treatment (orally administered antibiotic cocktails for 5 days followed with intraperitoneally administered clindamycin 1 day before challenge.)

### EXAMPLE 15

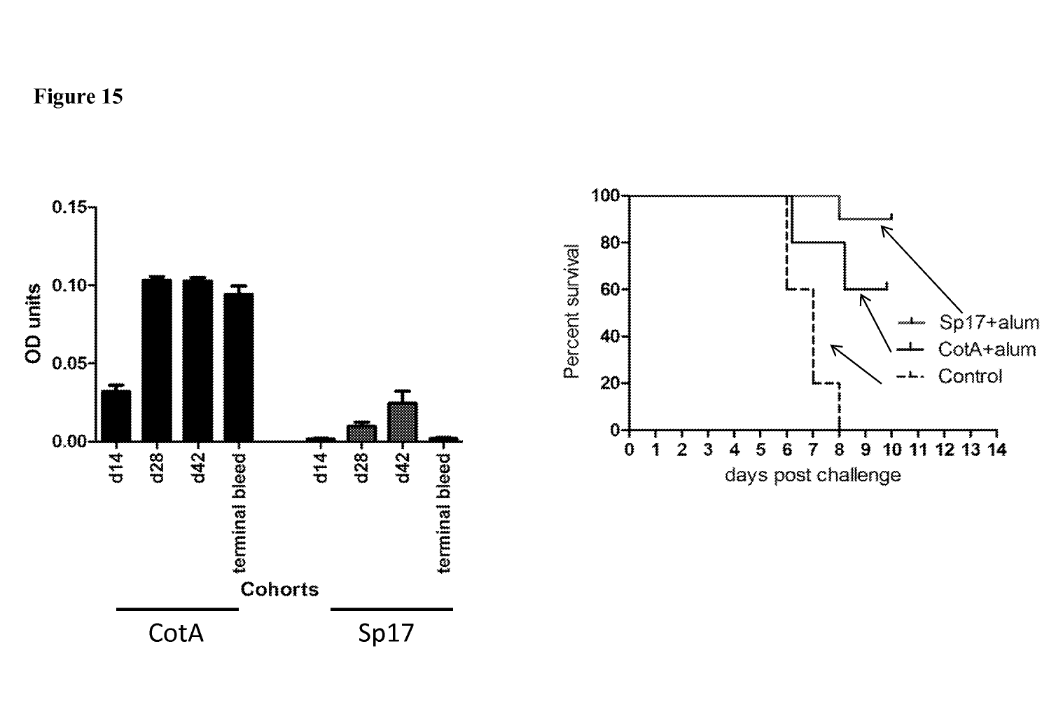

We i.p. immunized cohorts of 10 female, age-matched 8- to 10-week-old, C57BL/6 mice on days 0, 14 and 28 with 25 ug of recombinant CotA or Spl7 proteins adjuvanted with alum. Both proteins were able to mount a strong serum IgG immune response in the immunized mice. We challenged all cohorts of immunized mice and the control cohort two weeks post last immunization with C. difficile UK1. The cohort of mice that received 25 ug of Spl7 had protective efficacy of 90%, with 9 out of 10 mice surviving challenge by C. difficile UK1 (FIGS. 15a and b). Cohort of mice that received 25 ug of CotA had a protective efficacy of 60%. Control mice and the 5 immunized mice succumbed to challenge between days 6 and 8 post-challenge.

The following representative polypeptide sequences are presented to illustrate specific embodiments but are not intended to be limiting.

| SEQ ID NO: 1 | |
|---|---|
| (CD1067; GenBank ID YP_001087551) | |
| 1 | mqdykknkrr mmnqpmstmn eeevytdein sedmrgfkks hhhngcntdn kcechddcnp |
| 61 | cnpcnpckpn pcnpckpnpc ddncgchdnc kcdcepcemd sdecfenkcg peccnpispr |
| 121 | nfsvsnavpf aieanrifdt mqfqtftdat gpngepltfe tevvevfgsv psagqasvti |
| 181 | ekiclsndgi vidtgmttle dfdldplgdi vgrncettfe favcgernse ccrqgkgksv |
| 241 | aykqrgltva vrnlvlelrg rcgctefval afpavraggg ckrrvdyvef tfntlsapic |
| 301 | lpadgravtl rqeyqtnltv dcigksilkl ecneccepfy eliipndidl vlclqetvst |
| 361 | liseqivvla spnpiqprlv dtfskvcdfs qcgpnhgsgk pschr |
| SEQ ID NO: 2 | |
| (CD BclA1; GenBank ID YP_001086801.1) | |
| 1 | mrniilylnd dtfiskkypd knfsnldycl igskcsnsfv keklitffkv ripdilkdks |
| 61 | ilkaelfihi dsnknhifke kvdieikris eyynlrtitw ndrvsmenir gylpigisdt |
| 121 | snyiclnitg tikawamnky pnyglalsln ypyqilefts srgcnkpyil vtfedriidn |
| 181 | cypkcecppi ritgpmgprg atgstgpmgv tgptgstgat gsigptgptg ntgatgsigp |
| 241 | tgvtgptgst gatgsigptg vtgptgntgv tgsigptgat gptgntgvtg sigptgvtgp |
| 301 | tgntgeigpt gatgptgvtg sigptgatgp tgeigptgat gatgsigptg atgptgatgv |
| 361 | tgeigptgei gptgatgptg vtgsigptga tgptgatgei gptgatgptg vtgsigptga |
| 421 | tgptgatgei gptgatgptg vtgeigptga tgptgntgvt geigptgatg ptgntgvtge |
| 481 | igptgatgpt gvtgeigptg ntgatgsigp tgvtgptgat gsigptgatg atgvtgptgp |
| 541 | tgatgnssqp vanflvnaps pqtlnngdai tgwqtiigns ssitvdtngt ftvqengvyy |
| 601 | isvsvalqpg sssinqysfa ilfpilggkd laglttepgg ggvlsgyfag flfggttfti |
| 661 | nnfssttvgi rngqsagtaa tltifriadt vmt |
| SEQ ID NO: 3 | |
| (CD SleC; GenBank ID YP_001087027.1) | |
| 1 | mqdgfltvsi idatnnrpiq navvniysms ngsqssstly qnlrsnesgq vtglvlpapd |
| 61 | vdyslqpsdv rpysqyivea iadgyetvvi egtqllatie arqgvpmspr trskrsfsrq |
| 121 | selifdigeh tlygtyppki pesnlkplpp ptgfvvldnp vvpefivvhd glpedssapn |
| 181 | ywipfkeyik niasseiyst wpeqtiyanv iaiisftlnr vftewyrnkg ynftitstta |
| 241 | ydhkfinnrn lfepinvvvd aifntfikrp ptsrqpllaq ycdgqksqcp dqmtqwgskd |
| 301 | lgdqgydyes ilryfygdei vferapivsg vpvsfpgttl qvgssgqyvr tiqnqlnais |
| 361 | nsypavpkvi edgiygtdte navkifqgif glpqsgvvdf ktwyeisrvy vattriasln |
| 421 | pli |
| SEQ ID NO: 4 | |
| (CD CotA; YP_001088114.1) | |
| 1 | mennkcredf rftqeyeedy pntneryyen yqvadryyny pnkykepkik qccckksmre |
| 61 | alellrydal rpfvnfnqfa fisdffivga nlvgidlsap pkdnlsgldg tferfsacnc |
| 121 | dlidiagrvs ypipvpltle glintigtip gvaelialid avipptidlg aildailaai |
| 181 | idfilaastp lanvdlaslc nlkavafdit padyedfias lgyyldkkhy kecncncdcd |
| 241 | dcccnkgild nlymsninnq vtvvagslvl tgvevlgkkn dvivlgnsnd sriyfvcvds |
| 301 | idyia |
| SEQ ID NO: 5 | |
| (CD Sp17; YP_001088081.1) | |
| 1 | menkkcysed wyergestak wfqndreeye reaydedrer rgsncgcsds genrprncer |
| 61 | frreaeirer eareafcess ekkkealaye cearklweea ekywdeysky nykgieylae |
| 121 | aarlfdegme cearrngnng gnnnncchkc hkcncnccrk |
| SEQ ID NO: 6 | |
| (CD FliC; GenBank AAD46086.1) | |
| 1 | mrvntnvsal iannqmgrnv naqsksmekl ssgvrikraa ddaaglaise kmraqikgld |
| 61 | qagrnvqdgi svvqtaegal eetgnilqrm rtlsvqssne tntaeerqki adellqlkde |
| 121 | verisssief ngkklldgss teirlqvgan fgtnvagtsn nnneikvalv ntssimskag |
| 181 | itsstiasln adgtsgtnaa kqmvssldva lkelntsrak lgaqqnrles tqnnlnntie |
| 241 | nvtaaesrir dtdvasemvn lskmnilvqa sqsmlaqanq qpqgvlqllg s |
| SEQ ID NO: 7 | |
| (CD FliD GenBank ZP_05349430) | |
| 1 | mssispirvt glsgnfdmeg iieasmirdk ekvdkakqeq qivkwkqeiy rnviqeskdl |
| 61 | ydkylsvnsp nsivsekays stritssdes iivakgsaga ekinyqfays qmaepakfti |
| 121 | klnssepivq qfppnasgas sltigdvnip iseqdttsti vskinslcad ndikasysem |
| 181 | tgeliisrkq tgsssdinlr vigndnlaqq iandngitfi ndaggnkvan vygknleadv |
| 241 | tdehgrvthi skeqnsfnid nidynvnskg takltsvtdt eeavknmqaf vddynklmdk |
| 301 | vyglvttkkp kdyppltdaq kedmtteeie kwekkakegi lrnddelrgf vediqsaffg |
| 361 | dgkniialrk lginesenyn kkgqisfnad tfskalidds dkvyktlagy ssnyddkgmf |
| 421 | eklkdivyey sgsstsklpk kagiektasa senvyskqia eqernisrlv ekmndkekrl |
| 481 | yakysalesl lnqyssqmny fsqaqgn |
| SEQ ID NO: 8 | |
| (CD Toxin A GenBank AAA23283.1) | |
| 1 | msliskeeli klaysirpre neyktiltnl deynklttnn nenkylqlkk lnesidvfmn |
| 61 | kyktssrnra lsnlkkdilk eviliknsnt spveknlhfv wiggevsdia leyikqwadi |
| 121 | naeyniklwy dseaflvntl kkaivesstt ealqlleeei gnpqfdnmkf ykkrmefiyd |
| 181 | rqkrfinyyk sginkptvpt iddiikshlv seynrdetvl esyrtnslrk insnhgidir |
| 241 | anslfteqel lniysgelln rgnlaaasdi vrllalknfg gvyldvdmlp gihsdlfkti |
| 301 | srpssigldr wemikleaim kykkyinnyt senfdkldqq lkdnfkliie sksekseifs |
| 361 | klenlnvsdl eikiafalgs vinqaliskq gsyltnlvie qvknryqfln qhlnpaiesd |
| 421 | nnftdttkif hdslfnsata ensmfltkia pylqvgfmpe arstislsgp gayasayydf |
| 481 | inlqentiek tlkasdlief kfpennlsql teqeinslws fdqasakyqf ekyvrdytgg |
| 541 | slsedngvdf nkntaldkny llnnkipsnn veeagsknyv hyiiqlqgdd isyeatcnlf |
| 601 | sknpknsiii qrnmnesaks yflsddgesi lelnkyripe rlknkekvkv tfighgkdef |
| 661 | ntsefarlsv dslsneissf ldtikldisp knvevnllgc nmfsydfnve etypgkllls |
| 721 | imdkitstlp dvnknsitig anqyevrins egrkellahs gkwinkeeai msdlsskeyi |
| 781 | ffdsidnklk aksknipgla sisediktll ldasvspdtk filnnlklni essigdyiyy |
| 841 | eklepvknii hnsiddlide fnllenvsde lyelkklnnl dekylisfed isknnstysv |
| 901 | rfinksnges vyvetekeif skysehitke istiknsiit dvngnlldni qldhtsqvnt |
| 961 | lnaaffiqsl idyssnkdvl ndlstsvkvq lyaqlfstgl ntiydsiqlv nlisnavndt |
| 1021 | invlptiteg ipivstildg inlgaaikel ldehdpllkk eleakvgvla inmslsiaat |
| 1081 | vasivgigae vtifllpiag isagipslvn nelilhdkat svvnyfnhls eskkygplkt |
| 1141 | eddkilvpid dlviseidfn nnsiklgtcn ilameggsgh tvtgnidhff sspsisship |
| 1201 | slsiysaigi etenldfskk immlpnapsr vfwwetgavp glrslendgt rlldsirdly |
| 1261 | pgkfywrfya ffdyaittlk pvyedtniki kldkdtrnfi mptittneir nklsysfdga |
| 1321 | ggtyslllss ypistninls kddlwifnid nevreisien gtikkgklik dvlskidink |
| 1381 | nkliignqti dfsgdidnkd ryifltceld dkisliiein lvaksyslll sgdknylisn |
| 1441 | lsntiekint lgldskniay nytdesnnky fgaisktsqk siihykkdsk nilefyndst |
| 1501 | lefnskdfia edinvfmkdd intitgkyyv dnntdksidf sislvsknqv kvnglylnes |
| 1561 | vyssyldfvk nsdghhntsn fmnlfldnis fwklfgfeni nfvidkyftl vgktnlgyve |
| 1621 | ficdnnknid iyfgewktss skstifsgng rnvvvepiyn pdtgedists ldfsyeplyg |
| 1681 | idryinkvli apdlytslin intnyysney ypeiivlnpn tfhkkvninl dsssfeykws |
| 1741 | tegsdfilvr yleesnkkil qkirikgils ntqsfnkmsi dfkdikklsl gyimsnfksf |
| 1801 | nseneldrdh lgfkiidnkt yyydedsklv kglininnsl fyfdpiefnl vtgwqtingk |
| 1861 | kyyfdintga altsykiing khfyfnndgv mqlgvfkgpd gfeyfapant qnnniegqai |
| 1921 | vyqskfltln gkkyyfdnns kavtgwriin nekyyfnpnn aiaavglqvi dnnkyyfnpd |
| 1981 | taiiskgwqt vngsryyfdt dtaiafngyk tidgkhfyfd sdcvvkigvf stsngfeyfa |
| 2041 | pantynnnie gqaivyqskf ltlngkkyyf dnnskavtgw gtidskkyyf ntntaeaatg |
| 2101 | wqtidgkkyy fntntaeaat gwqtidgkky yfntntaias tgytiingkh fyfntdgimq |
| 2161 | igvfkgpngf eyfapantda nniegqaily qnefltlngk kyyfgsdska vtgwriinnk |
| 2221 | kyyfnpnnai aaihlctinn dkyyfsydgi lqngyitier nnfyfdanne skmvtgvfkg |
| 2281 | pngfeyfapa nthnnniegq aivyqnkflt lngkkyyfdn dskavtgwqt idgkkyyfnl |
| 2341 | ntaeaatgwq tidgkkyyfn lntaeaatgw qtidgkkyyf ntntfiastg ytsingkhfy |
| 2401 | fntdgimqig vfkgpngfey fapantdann iegqailyqn kfltingkky yfgsdskavt |
| 2461 | glrtidgkky yfntntavav tgwqtingkk yyfntntsia stgytiisgk hfyfntdgim |
| 2521 | qigvfkgpdg feyfapantd anniegqair yqnrflylhd niyyfgnnsk aatgwvtidg |
| 2581 | nryyfepnta mgangyktid nknfyfrngl pqigvfkgsn gfeyfapant danniegqai |
| 2641 | ryqnrflhll gkiyyfgnns kavtgwqtin gkvyyfmpdt amaaagglfe idgviyffgv |
| 2701 | dgvkapgiyg |
| SEQ ID NO: 9 | |
| (CD Toxin B GenBank P18177.3). | |
| 1 | mslvnrkqle kmanvrfrtq edeyvailda leeyhnmsen tvvekylklk dinsltdiyi |
| 61 | dtykksgrnk alkkfkeylv tevlelknnn ltpveknlhf vwiggqindt ainyinqwkd |
| 121 | vnsdynvnvf ydsnaflint lkktvvesai ndtlesfren lndprfdynk ffrkrmeiiy |
| 181 | dkqknfinyy kaqreenpel iiddivktyl sneyskeide lntyieesln kitqnsgndv |
| 241 | rnfeefknge sfnlyeqelv erwnlaaasd ilrisalkei ggmyldvdml pgiqpdlfes |
| 301 | iekpssvtvd fwemtkleai mkykeyipey tsehfdmlde evqssfesvl asksdkseif |
| 361 | sslgdmeasp levkiafnsk giinqglisv kdsycsnliv kqienrykil nnslnpaise |
| 421 | dndfntttnt fidsimaean adngrfmmel gkylrvgffp dvkttinlsg peayaaayqd |
| 481 | llmfkegsmn ihlieadlrn feisktnisq steqemaslw sfddarakaq feeykrnyfe |
| 541 | gslgeddnld fsqnivvdke yllekissla rssergyihy ivqlqgdkis yeaacnlfak |
| 601 | tpydsvlfqk niedseiayy ynpgdgeiqe idkykipsii sdrpkikltf ighgkdefnt |
| 661 | difagfdvds lsteieaaid lakedispks ieinllgcnm fsysinveet ypgklllkvk |
| 721 | dkiselmpsi sqdsiivsan qyevrinseg rrelldhsge winkeesiik disskeyisf |
| 781 | npkenkitvk sknlpelstl lqeirnnsns sdieleekvm lteceinvis nidtqiveer |
| 841 | ieeaknitsd sinyikdefk liesisdalc dlkqqneled shfisfedis etdegfsirf |
| 901 | inketgesif vetektifse yanhiteeis kikgtifdtv ngklvkkvnl dtthevntln |
| 961 | aaffiqslie ynsskeslsn lsvamkvqvy aqlfstglnt itdaakvvel vstaldetid |
| 1021 | llptlseglp iiatiidgvs lgaaikelse tsdpllrqei eakigimavn lttattaiit |
| 1081 | sslgiasgfs illvplagis agipslynne lvlrdkatkv vdyfkhvslv etegvftlld |
| 1141 | dkimmpqddl viseidfnnn sivlgkceiw rmeggsghtv tddidhffsa psityrephl |
| 1201 | siydvlevqk eeldlskdlm vlpnapnrvf awetgwtpgl rslendgtkl ldrirdnyeg |
| 1261 | efywryfafi adalittlkp ryedtnirin ldsntrsfiv piitteyire klsysfygsg |
| 1321 | gtyalslsqy nmginielse sdvwiidvdn vvrdvtiesd kikkgdlieg ilstlsieen |
| 1381 | kiilnshein fsgevngsng fvsltfsile ginaiievdl lsksykllis gelkilmlns |
| 1441 | nhiqqkidyi gfnselqkni pysfvdsegk engfingstk eglfvselpd vvliskvymd |
| 1501 | dskpsfgyys nnlkdvkvit kdnvniltgy ylkddikisl sltlqdekti klnsvhldes |
| 1561 | gvaeilkfmn rkgntntsds lmsflesmni ksifvnflqs nikfildanf iisgttsigq |
| 1621 | feficdendn iqpyfikfnt letnytlyvg nrqnmivepn ydlddsgdis stvinfsqky |
| 1681 | lygidscvnk vvispniytd einitpvyet nntypevivl danyinekin vnindlsiry |
| 1741 | vwsndgndfi lmstseenkv sqvkirfvnv fkdktlankl sfnfsdkqdv pvseiilsft |
| 1801 | psyyedglig ydlglvslyn ekfyinnfgm mvsgliyind slyyfkppvn nlitgfvtvg |
| 1861 | ddkyyfnpin ggaasigeti iddknyyfnq sgvlqtgvfs tedgfkyfap antldenleg |
| 1921 | eaidftgkli ideniyyfdd nyrgavewke ldgemhyfsp etgkafkgln qigdykyyfn |
| 1981 | sdgvmqkgfv sindnkhyfd dsgvmkvgyt eidgkhfyfa engemqigvf ntedgfkyfa |
| 2041 | hhnedlgnee geeisysgil nfnnkiyyfd dsftavvgwk dledgskyyf dedtaeayig |
| 2101 | lslindgqyy fnddgimqvg fvtindkvfy fsdsgiiesg vqniddnyfy iddngivqig |
| 2161 | vfdtsdgyky fapantvndn iygqaveysg lvrvgedvyy fgetytietg wiydmenesd |
| 2221 | kyyfnpetkk ackginlidd ikyyfdekgi mrtglisfen nnyyfnenge mqfgyinied |
| 2281 | kmfyfgedgv mqigvfntpd gfkyfahqnt ldenfegesi nytgwldlde kryyftdeyi |
| 2341 | aatgsviidg eeyyfdpdta qlvise |

Although the invention has been described in detail for the purposes of illustration, it is understood that such detail is solely for that purpose, and variations can be made therein by those skilled in the art without departing from the spirit and scope of the invention which is defined by the following claims.

We claim:

1. A method for prophylaxis and/or therapy of Clostridium difficile (C. difficile) infection comprising administering to a subject in need thereof an effective amount of a pharmaceutical composition comprising at least one Clostridium difficile (C. difficile) polypeptide, wherein the C. difficile polypeptide is selected from the group consisting of CD1067, BclA1, SleC, CotA, Spl7, and combinations thereof.

2. The method of claim 1, wherein the pharmaceutical composition comprises at least the CD1067 C. difficile polypeptide.

3. The method of claim 2, wherein the subject is at risk for contracting the C. difficile infection, and wherein the administering results in less diarrhea in the subject relative to an individual who has the C. difficile infection but did not receive the pharmaceutical composition.

4. The method of claim 3, wherein the subject is exposed to C. difficile and/or C. difficile spores.

5. The method of claim 2, wherein the subject has the C. difficile infection, and wherein the subject has less diarrhea relative to an individual who has the C. difficile infection but did not receive the pharmaceutical composition.

6. The method of claim 2, wherein the subsequent to the administering the subject produces neutralizing antibodies that react with specificity to at least the CD1067 C. difficile polypeptide.

7. The method of claim 2, wherein C. difficile colonization of the subject is inhibited or prevented.

8. The method of claim 2, wherein subsequent to the administering C. difficile infection relapse in the individual is inhibited or prevented.

9. A pharmaceutical composition comprising a Clostridium difficile (C. difficile) polypeptide selected from the group consisting of CD1067, BclA1, SleC, CotA, Spl7, and combinations thereof, for use in prophylaxis and/or therapy of C. difficile infection.

10. The pharmaceutical composition of claim 9 comprising the CD1067 polypeptide.

11. The pharmaceutical composition of claim 9, comprising at least one other C. difficile polypeptide selected from the group consisting of CD toxin A (CDA), CD toxin B (CDB), FliC, and FliD, and combinations thereof.

12. The pharmaceutical composition of claim 10 further comprising at least one of the BclA1, SleC, CotA, and Spl7 polypeptides.

13. The pharmaceutical composition of claim 12, further comprising at least one of the CDA, CDB, FliC or FliD polypeptides.

14. The pharmaceutical composition of claim 9 comprising at least two of the C. difficile polypeptides, wherein the at least two of the C. difficile polypeptides are present in a single fusion protein.

15. The pharmaceutical composition of claim 14, wherein at least one of the at least two polypeptides is a CD1067 polypeptide.

16. An article of manufacture comprising a pharmaceutical composition comprising packaging, and comprising a Clostridium difficile (C. difficile) polypeptide selected from the group consisting of CD1067, BclA1, SleC, CotA, Spl7, and combinations thereof, in a sealed container, wherein the pharmaceutical composition is a cell free composition, and wherein the packaging comprises printed material, and wherein the printed material provides an indication that the pharmaceutical composition is to be used for prophylaxis and/or treatment of C. difficile infection.

17. The article of manufacture of claim 16, wherein the container comprises at least the CD1067 polypeptide.
